(19) United States
(12) Patent Application Publication
(10) Pub. No.: US 2014/0127252 A1
(43) Pub. Date: May 8, 2014

(54) TULAREMIA-TAA/CD40L VACCINE

(71) Applicant: Albert B. Deisseroth, Potomac, MD (US)

(72) Inventor: Albert B. Deisseroth, Potomac, MD (US)

(73) Assignee: MICROVAX, LLC, Manassas, VA (US)

(21) Appl. No.: 14/154,710

(22) Filed: Jan. 14, 2014

Related U.S. Application Data
(63) Continuation-in-part of application No. 11/593,458, filed on Nov. 6, 2006.
(60) Provisional application No. 61/754,984, filed on Jan. 22, 2013.

(51) Int. Cl. A61K 39/02

(52) U.S. Cl. CPC A61K 39/0208
USPC 424/190.1

(57) ABSTRACT

A Tularemia vaccine proposes to induce antibodies which through their binding to the target proteins of the vaccine strategy will inactivate the proteins essential to mediating the secretory function of the pilus proteins of the “type IV pilus proteins” or Tfp proteins, thereby impairing the formation of the pilus proteins on the surface of the FT which are needed for binding and entry of the FT into macrophages, and will inhibit the secretion of several factors of FT which are necessary for the virulence of the FT, and which are missing in attenuated strains of FT used in current attenuate strain vaccination strategies. The vaccine may be a mixture of DNA plasmids encoding TAA/ecdCD4OL protein vaccines in which the TAA is comprised of fragments of the following Tularemia proteins: Tfp, pilA, pilC, pilT, and pilQ,

### CROSS-REFERENCE TO RELATED APPLICATIONS

This application claims priority and the benefit under 35 U.S.C. paragraph 119(e) of U.S. provisional patent application Ser. No. 61/754,984 filed on Jan. 22, 2013 which, including all figures and tables, are incorporated herein by reference in its entirety. This application is also a continuation-in-part of application Ser. No. 11/593,458, filed on Nov. 6, 2006 which, including all figures and tables, is incorporated herein by reference in its entirety.

### FIELD OF THE INVENTION

The present invention relates generally to the field of vaccines and more specifically to a vaccine for Tularemia, a highly infectious bacterial disease.

### BACKGROUND OF THE INVENTION

Tularemia. Francisella tularensis (FT) is a facultative intracellular (primarily macrophages) gram-negative coccobacillus which is highly infectious (1) bacterial cells can constitute a lethal dose) and can be acquired through the dermal, GI and pulmonary routes. Its reservoir in North America is in rabbits. Its uptake and replication in macrophages depends on several virulence factors some of which are critical to its entry into human cells, to replication within human cells, to the suppression of the innate and adaptive immune response in human cells, and to the release of the infectious organisms produced within macrophages (1-4). Its virulence and rapid spread within the infected individual lead to the incapacitation of that individual due to high fever, lethargy, reddening of the face and eyes, swelling of the lymph nodes (which may suppurate) and ultimately multi-organ failure. The mortality exceeds 50% if not treated with aminoglycoside antibiotics, and over 70% of patients dying of tularemia have lesions of pulmonic tularemia (5-7).

Tularemia is transmitted by tick or deer fly bites, inhalation of aerosols of the fluids of infected animals or ingestion of the meat of infected animals.

The clinical picture of Tularemia includes high fever, skin lesions (eschar surrounded by erythema), reddening of the face and eyes, lymphadenopathy (which may suppurate), ulcerations in the pharyngeal mucosa, lethargy, and death due to multi-organ failure. Following acquisition of the FT infection through aerosols, ingestion of infected water or meat, or transdermal penetration through a defect in the skin or GI tract, the bacterial cells enter the macrophages in which they replicate. Seven to ten days after the initial infection, the accumulation of live FT organisms leads to the death of the infected macrophages and to the subsequent release of the bacterial organisms. Ultimately, there is spread to lymph nodes, visceral organs and the blood.

Biological Warfare and the Development of Vaccines for Tularemia. FT was first reported to be associated with outbreaks in Canaan in 1715 BC. There was a prolonged outbreak in the Mediterranean in the 14^{th} century BC. FT was said to have been used successfully as an offensive weapon of biological warfare during World War II by the Russians against the German invaders during the siege of Stalingrad (8). Both Russia and the United States have developed strains of FT which are resistant to most antibiotics (8). During World War II, the USA amassed a stockpile of FT dry powder which was estimated to be sufficient to generate infections in 2.5 million people and to kill 200,000 (8). The United States has spent 100 million dollars in the past 10 years on vaccine development, most of which was devoted to attenuated live vaccines (see Table 1 in reference 8). Most of these vaccines are only partially protective, and these vaccines are least protective against inhalation of FT in aerosols (8).

Development of Tularemia Vaccines in Russia. As stated above, Tularemia was used by the Russian military against the German invaders during the siege of Stalingrad in World War II. The “SCHU S4” strain (otherwise known as Agent UL) was prepared for use in biological warfare programs. Many of these agents were resistant to streptomycin. These agents have a projected mortality of 60%. Other strains (e.g. Agent 425) were designed to incapacitate, rather than to kill.

Russian investigations in the 1950s, showed that immunization through inhalation of dried but viable attenuated strains of Tularemia, is possible. Systemic reactions were generated in a minority of 138 volunteers who inhaled between 700,000 and 7 million organisms. Response was measured by serology and skin tests (7).

The United States acquired an attenuated Tularemia vaccine strain (which became known as the live vaccine strain (LVS)) from Russia in the 1950s (8). This attenuated strain was subcloned and a sub-clone strain was developed which protected mice against the tularensis subspecies (8). In the United States, vaccination with the LVS has conferred partial immunity against challenge by the respiratory route with 200-2000 virulent organisms. But this could be overcome by increasing the dose of the challenge (5-7).

Fort Dietrick “Operation Whitecoat” Experience (7): The LVS was not associated with the formation of bubos or pneumonitis following percutaneous administration, and therefore by definition is a live attenuated vaccine strain. Five groups of normal volunteers were administered doses of the LVS as aerosols in doses ranging from 10,000 to 100 million organisms. After 10,000 CFU, the volunteers noted sore throat, cough, and cervical node swelling (pea size) without pulmonary infiltrates (7). Following administration of 100 million CFU, the reactions were more severe: headache, coryza, chest pain, temperatures >100 F, and transient infiltrates of the lungs (7).

Results of Vaccination with Attenuated Strains of Tularemia. The immune response to the vaccination was measured by the level of agglutinating antibodies to Tularemia which formed between 2-3 weeks after vaccination. Protection against pulmonary challenge with the SCHU S4 strain (25,000 CFU) generated 50-70% protection for 18 months (7). Protection was afforded to the transdermal administration of the SCHU S4 strain (7). The lack of any surrogate measurement that could be used to define successful immunization, and the absence of any understanding of the mechanism of attenuation have prevented licensing of this LVS for administration to the public at large (8).

The reason why the attenuated strains have not been licensed in the United States, is that there is a significant possibility that the attenuated vaccine will revert to a more fulminant and virulent strain, and the fact that the molecular basis for the attenuation is incompletely understood (8).

Knowledge of the Biology of FT Opens the Door to Recombinant Vaccines. Due to the development of a more detailed understanding of some of the mechanisms through which FT can bind to and enter macrophages, it has now become possible to consider recombinant vaccine strategies for Tularemia. The new information bearing on the molecular mechanisms FT uses relate to the adherence and uptake of FT by macrophages (in which the FT replicate), and to the secretion of its virulence factors. In addition, the increased understanding of the molecular basis of action of virulence factors which affect the following FT functions: uptake, intracellular replication, secretion of FT proteins and release of live FT cells by infected macrophages (9-13), now make it possible to design immunological counter measures against these factors. Finally, the development of a new and potent recombinant vaccine strategy by Applicant's laboratory (14-24), provides a molecular vehicle through which to develop a recombinant vaccine for Tularemia.

Development by Applicant of the TAA/ecdCD40L Vaccine Platform. The vaccine is based on the attachment of a fragment of a target associated antigen (TAA) fused to the extracellular domain (ecd) of the potent immunostimulatory signal CD40 ligand (CD40L). The vaccine can be administered either as a TAA/ecdCD40L protein, or as an expression vector encoding the TAA/ecdCD40L such as virus including the adenoviral vector: Ad-sig-TAA/ecdCD40L vector, or other viral vectors, or a plasmid DNA expression vector encoding the TAA/ecdCD40L protein. The vaccine can be also administered as a vector prime followed in 7 and 21 days with sc injections of the TAA/ecdCD40L protein vaccine. This vaccine platform was developed by Applicant's laboratory (14-24) to overcome the following problems: weak immunogenicity of the target antigens, qualitative or quantitative defects of CD4 helper T cells, defective response in immunodeficient individuals including the older aged population due to diminished expression of CD40L in activated CD4 helper T cells, and/or low levels of presentation of target antigens on Class I or II MHC in dendritic cells (DCs). The CD40L is important for the expansion of antigen specific CD8 effector T cells and antigen specific B cells in response to vaccination.

The activated TAA loaded DCs then migrate to the regional lymph nodes (14, 16) where they can activate and induce expansion of the TAA specific CD8⁺ effector T cells. These antigen specific CD8⁺ effector cells become increased in number in the lymph nodes (14, 16), and they then egress from the lymph nodes into the peripheral blood. The antigen specific CD8+ effector T cells exit the intravascular compartment and enter into the extra-vascular sites of inflammation or infection (18). In addition to showing that this vaccine increases the levels of the antigen specific CD8⁺ effector T cells in the sites of inflammation or infection (18), the Applicant's laboratory has shown that the activation and expansion of the B cells by the TAA/ecdCD40L protein increases the levels of the TAA specific antibodies (including neutralizing antibodies against viral antigens) in the serum (18, 21, 22, 24).

Activation of DCs by TAA/ecdCD40L Vaccine. The activated TAA loaded DCs then migrate to the regional lymph nodes (14, 18) where they can activate and induce expansion of the TAA specific CD8⁺ effector T cells. These antigen specific CD8⁺ effector cells become increased in number in the lymph nodes (14, 18), and they then egress from the lymph nodes into the peripheral blood. The antigen specific CD8 effector T cells exit the intravascular compartment and enter into the extra-vascular sites of inflammation or infection (21, 22 and 24). In addition to showing that this vaccine increases the levels of the antigen specific CD8⁺ effector T cells in the sites of inflammation or infection (24), the Applicant's laboratory has shown that the activation and expansion of the B cells by the TAA/ecdCD40L protein increases the levels of the TAA specific antibodies including neutralizing antibodies against viral antigens in the serum (21, 22 and 24).

Impact of Attachment of TAA to CD40L. The attachment of fragments of the TAA to the CD40L accomplishes two objectives: 1. The binding of the TAA/ecdCD40L protein to the CD40 receptor on the DCs as well as on the B cells and T cells, activating these cells thereby promoting a potent immune response (14, 16 and 18); 2. Once the TAA/ecdCD40L protein is engaged on the CD40 receptor of the DC, the entire TAA/ecdCD40L protein is internalized into the DC in a way that allows Class I as well as Class II MHC presentation of the TAA (14, 18).

Modes of Administration. There are four versions of this vaccine: 1. One in which the TAA/ecdCD40L transcription unit is embedded in a replication incompetent adenoviral vector (Ad-sig-TAA/ecdCD40L); 2. One in which the vector is used as an initial priming injection, followed by two sc injections of the TAA/ecdCD40L protein; 3. One in which the vaccine consists solely of the TAA/ecdCD40L protein; and 4. One in which the TAA/ecdCD40L is inserted into a plasmid DNA expression vector. The TAA is connected through a linker to the aminoterminal end of the ecd of the potent immunostimulatory signal CD40L.

Historical Summary of the Development of the TAA/ecdCD40L Vaccine Platform the Development of Which the Applicant Participated as a Co-Inventor

Previous Vaccines. Vaccines have been described that include an adenoviral expression vector encoding a fusion protein that includes an antigen fused to CD40 ligand. See, e.g., U.S. Patent Application Publication US 2005-0226888 (Application Ser. No. 11/009,533) titled “Methods for Generating Immunity to Antigen,” filed Dec. 10, 2004.

### SUMMARY OF THE INVENTION

Applicant's Tularemia vaccine proposes to induce antibodies which through their binding to the target proteins of the vaccine strategy will inactivate the proteins essential to mediating the secretory function of the pilus proteins of the “type IV pilus proteins” or Tfp proteins, thereby impairing the formation of the pilus proteins on the surface of the FT which are needed for binding and entry of the FT into macrophages, and will inhibit the secretion of several factors of FT which are necessary for the virulence of the FT, and which are missing in attenuated strains of FT used in current attenuate strain vaccination strategies.

In terms of composition of matter, Applicant's vaccine is a recombinant vaccine rather than a live attenuated vaccine, and is composed of fragments of FT proteins which have never been linked to immunostimulatory proteins to promote a potent adaptive immune response.

### OBJECTIVES OF THE INVENTION

Any one or more major objectives for a critically needed successful Tularemia vaccine, not heretofore developed, include:
1. preventing the infection of human cells (macrophages) by FT and its subsequent replication within these cells by inducing high titers of antibodies to the proteins involved in the function of the pilus protein (Tfp) which mediates the binding and uptake of FT into macrophages which is necessary for the replication of FT,
2. a reduction in virulence of FT by binding of antibodies to the Tfp proteins involved in the secretion of the virulence factors, converting FT protein fragments which are weak immunogens into potent immunogens,
3. protecting vaccinated individuals from FT infection during the first year of the vaccination,
4. providing long term memory for the immune response induced by the vaccination, and
5. avoiding significant side effects to the extent possible.

In addition, it is an additional objective of Applicant's vaccine to propose to induce antibodies which through their binding to the target proteins of the vaccine strategy will inactivate the proteins essential to mediating the secretory function of the pilus proteins of the “type IV pilus proteins” or Tfp proteins, thereby impairing the formation of the pilus proteins on the surface of the FT which are needed for binding and entry of the FT into macrophages, and will inhibit the secretion of several factors of FT which are necessary for the virulence of the FT, and which are missing in attenuated strains of FT used in current attenuate strain vaccination strategies.

It is yet a further objective in that the Tularemia vaccine is a recombinant vaccine rather than a live attenuated vaccine, and is composed of fragments of FT proteins which have never been linked to immunostimulatory proteins to promote a potent adaptive immune response.

Other objectives will become clear pursuant to the following detailed description.

### DETAILED DESCRIPTION

Definitions: The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context.

As used herein, the terms “antigen” or “antigenic factors” refers broadly to any antigen to which a human, mammal, bird or other animal can generate an immune response. The terms “antigen” or “antigenic factors” as used herein refers broadly to a molecule that contains at least one antigenic determinant to which the immune response may be directed. The immune response may be cell-mediated, humoral or both. As is well known in the art, an antigen may be protein, carbohydrate, lipid, or nucleic acid or any combinations of these biomolecules. As is also well known in the art, an antigen may be native, recombinant or synthetic. For example, an antigen may include non-natural molecules such as polymers and the like. Antigens include both self-antigens and non-self antigens. As used herein, “antigenic determinant” (or epitope) refers to a single antigenic site on an antigen or antigenic factor; it is a minimal portion of a molecule that recognized by the immune system, specifically by antibodies, B cells or T cells. Antigenic determinants may be linear or discontinuous.

“Pharmaceutically acceptable” in the context of the present invention means a pharmaceutical composition that is generally safe, non-toxic and biologically acceptable for veterinary and human pharmaceutical use. Preferred compositions of this invention are intended for humans or animals.

The phrase “an effective amount” in reference to administering the fusion protein or an expression vector encoding that protein is an amount that results in an increase in the immune response as measured by an increase in T cell activity or antibody production.

The fusion protein complex mixture recited herein may be formulated with an adjuvant to enhance the resulting immune response. As used herein, the term “adjuvant” in the context of the instant invention means a chemical that, when administered with the expression vector or the fusion protein, enhances the immune response. An adjuvant is distinguished from a carrier protein in that the adjuvant is not chemically coupled to the antigen. Adjuvants are well known in the art and include, but not limited to, mineral oil emulsions (U.S. Pat. No. 4,608,251) such as Freund's complete or Freund's incomplete adjuvant (Freund, Adv. Tuberc. Res. 7:130 (1956); Calbiochem, San Diego Calif.), aluminum salts, especially aluminum hydroxide or ALHYDROGEL (approved for use in humans by the U.S. Food and Drug Administration), muramyl dipeptide (MDP) and its analogs such as [Thr¹]-MDP (Byersand Allison, Vaccine 5:223 (1987)), monophosphoryl lipid A (Johnson et al., Rev. Infect. Dis. 9:S512 (198)), and the like.

The term “vector” as used in this application contains a transcription unit (also known as an “expression vector”). It encompasses both viral and non-viral expression vectors that when administered in vivo can enter target cells and express an encoded protein. Viral vectors have evolved means to overcome cellular barriers and immune defense mechanisms. Viral vectors suitable for in vivo delivery and expression of an exogenous protein are well known in the art and include adenoviral vectors, adeno-associated viral vectors, retroviral vectors, vaccinia vectors, pox vectors, herpes simplex viral vectors, etc. Viral vectors are preferably made replication defective in normal cells. For example, see U.S. Pat. Nos. 6,669,942; 6,566,128; 6,794,188; 6,110,744 and 6,133,029.

On the other hand, nonviral gene carriers consistently exhibit significantly reduced transfection efficiency as they are hindered by numerous extra- and intracellular obstacles. Non-viral vectors for gene delivery comprise various types of expression vectors (e.g., plasmids) which are combined with lipids, proteins and other molecules (or combinations of thereof) in order to protect the DNA of the vector during delivery. Fusigenic non-viral particles can be constructed by combining viral fusion proteins with expression vectors as described. Kaneda, Curr Drug Targets (2003) 4(8):599-602. Reconstituted HVJ (hemagglutinating virus of Japan; Sendai virus)-liposomes can be used to deliver expression vectors or the vectors may be incorporated directly into inactivated HVJ particles without liposomes. See Kaneda, Curr Drug Targets (2003) 4(8):599-602. DMRIE/DOPE lipid mixture is useful as a vehicle for non-viral expression vectors. See U.S. Pat. No. 6,147,055. Polycation-DNA complexes also may be used as a nonviral gene delivery vehicle. See Thomas et al., Appl Microbiol Biotechnol (2003) 62(1):27-34. The vector can be administered parenterally, such as intravascularly, intravenously, intra-arterially, intramuscularly, subcutaneously, or the like. Administration can also be orally, nasally, rectally, transdermally or aerosol inhalation. The vectors may be administered as a bolus, or slowly infused. The vector is preferably administered subcutaneously.

The term “transcription unit” as used herein in connection with an expression vector means a stretch of DNA, that is transcribed as a single, continuous mRNA strand by RNA polymerase, and includes the signals for initiation and termination of transcription. For example, in one embodiment, a transcription unit of the invention includes nucleic acid that encodes from 5′ to 3′ a secretory signal sequence, an influenza antigen and CD40 ligand. The transcription unit is in operable linkage with transcriptional and/or translational expression control elements such as a promoter and optionally any upstream or downstream enhancer element(s). A useful promoter/enhancer is the cytomegalovirus (CMV) immediate-early promoter/enhancer. See U.S. Pat. Nos. 5,849,522 and 6,218,140.

The term “secretory signal sequence” (also known as “signal sequence,” “signal peptide,” leader sequence,” or leader peptide”) as used herein refers to a short peptide sequence, generally hydrophobic in charter, including about 20 to 30 amino acids that is synthesized at the N-terminus of a polypeptide and directs the polypeptide to the endoplasmic reticulum. The secretory signal sequence is generally cleaved upon translocation of the polypeptide into the endoplasmic reticulum. Eukaryotic secretory signal sequences are preferred for directing secretion of the exogenous gene product of the expression vector. A variety of suitable such sequences are well known in the art and include the secretory signal sequence of human growth hormone, immunoglobulin kappa chain, and the like. In some embodiments, the endogenous tumor antigen signal sequence also may be used to direct secretion.

The term “CD40 ligand” (CD40L) as used herein refers to a full length or portion of the molecule known also as CD154 or TNFS. CD40L is a type II membrane polypeptide having a cytoplasmic domain at its N-terminus, a transmembrane region and then an extracellular domain (ecd) at its C-terminus Unless otherwise indicated the full length CD40L is designated herein as “CD40L,” “wtCD40L” or “wtTmCD40L.” The nucleotide and amino acid sequence of CD40L from mouse and human is well known in the art and can be found, for example, in U.S. Pat. No. 5,962,406. Also, included within the meaning of CD40 ligand are variations in the sequence including, but not limited to, conservative amino acid changes and the like which do not alter the ability of the ligand to elicit an immune response in conjunction with the fusion protein of the invention.

The term “linker” as used or employed in this application with respect to the transcription unit of the expression vector refers to one or more amino acid residues between the carboxy terminal end of the antigen and the amino terminal end of CD40 ligand. The composition and length of the linker may be determined in accordance with methods well known in the art and may be tested for efficacy. (See, e.g. Arai et al. Protein Engineering, Vol. 4, No. 8, 529-532, August 2001). In certain embodiments of the present invention, the linker is from about 3 to about 15 amino acids long, more preferably from about 5 to about 10 amino acids long. However, longer or shorter linkers may be used or the linker may not be used at all. Longer linkers may be up to about 50 amino acids, or up to about 100 amino acids. One example of a linker well known in the art is a 15 amino acid linker consisting of three repeats of four glycines and a serine (i.e., [Gly₄Ser₃)].

The term “TAA” recited herein refers to a target associated antigen, which may, for example, be a viral antigen, a bacterial antigen, a tumor antigen, etc.

### Criteria for Selection of the Fragments of the Tularemia Proteins for the TAA/ecdCD40L Vaccines

The cDNA, as one embodiment, which encodes fragments selected from each of these Tularemia proteins will be attached to the cDNA encoding ecdCD40L to create a TAA/ecdCD40L plasmid transcription unit which will be inserted into an expression vector (viral or plasmid) for use in vaccination. The vaccine will be a mixture of these expression vectors (viral or plasmid) which will be administered IM at weekly intervals for three injections. Each of the Tularemia proteins listed above was chosen because it has been shown to play a major role in the binding and entry of the FT into macrophages, and/or be necessary for the virulence of the FT organisms.

The fragments of each of these proteins will be selected based on the following criteria:
a. Small enough so as not to disrupt the homotrimeric structure of the ecdCD40L;
b. Contain aminoacid domains which bind to and are recognized by Class I MHC;
c. Contain aminoacid domains which bind to and are recognized by Class II MHC;
d. TAA/ecdCD40L encoding expression vectors (viral or plasmid), which contain multiple fragments from separate proteins will be administered simultaneously to decrease the probability of immunological escape.

### Functional Targets of the Antigen Fragments Identified for the TAA/ecdCD40L Tularemia Vaccine

Antigen fragment functional targets for vaccine development include:
1. The Type IV pili proteins (Tfp): These proteins are surface fimbriae or pili which are important in adhesion to target host cells and secretion of exotoxins and other virulence proteins (25-29). This apparatus can affect surface motility, host cell adhesion, and natural transformation (12). The aminoterminal end of the fimbriae makes contact with the cellular receptors. Pilans from different bacterial species have the following highly conserved consensus amino acid sequence at the aminoterminal contact end: FTLIELMIVVAIIGILAAIALPAYQDYTARSQ (30). This sequence of amino acids will be attached to the amino terminal end of the ecdCD40L through a linker.
2. Pilan A protein (pilA): pilA is a Tfp protein which affects virulence of the SCHU S4 strain (13, 26). The surface localization of pilA on SCHU S4 is necessary for full virulence of SCHU S4 in the mouse infection model (13). The pilA protein is missing in the LVS attenuated strain of FT (13). pilA is required for natural transformation, and necessary for the formation of the Tpf pili apparatus (29).
3. Pilan C protein (pilC): pilC is a transmembrane protein encoded by FT (28) which is important in the secretion of the Tfp protein. Secretion of the Tfp is important for the virulence of FT (29) and of the SCHU S4 strain (13)
4. Pilan T protein (pilT): pilT is intact in most virulent strains of FT and is important for the virulence of those strains (13). It is an ATPase (13) and one of its known functions has to do with pilan retraction and twitching (30) but it is not clear that its ATPase function is relevant to its effect of the virulence of FT.
5. Pilan Q protein (pilQ); pilQ is important in the secretion of the Tfp protein and important for the virulence of SCHU S4 (13). The pilQ protein forms the pore on the surface of the FT bacterial cell through which the pilus apparatus is extruded, or through which virulence factors are secreted (30).

This group of proteins is believed to have never been combined together to form a recombinant vaccine for Tularemia nor have these proteins ever been attached to the ecdCD40L for purposes of vaccination. Moreover, the unique aspect of this invention is that the vaccine is designed to disrupt and prevent the formation and function of the pore of a secretory apparatus on the surface of the FT bacterial cell so as to prevent attachment of the bacterial cell to the target host cell, and to prevent the release of virulence factors from the bacterial cell.

Applicant has elected to employ Applicant's TAA/ecdCD40L vaccine platform in the following manner as a preferred embodiment, defining criteria for a Tularemia vaccine and which meets the above identified objectives:
1. The IM administration of a mixture of expression vectors (viral or DNA plasmids) encoding TAA/ecdCD40L protein vaccines in which the TAA is comprised of fragments of the following Tularemia proteins: Tfp, pilA, pilC, pilT, and pilQ, to induce very high titer levels of antibodies to these proteins resulting in a reduction of virulence of FT, and a prevention of the entry of FT into the macrophages into which it must enter to replicate.
2. The attachment of the following Tularemia protein fragments: Tfp, pilA, pilC, pilT, and pilQ to the aminoterminal end of the ecdCD40L, to convert these weak immunogens into potent immunogens and thereby induce very high titer levels of antibodies to these peptides such that the FT organism no longer binds to nor penetrates the macrophage.
3. The administration of expression vectors (viral or DNA plasmid) vaccines which encode the following Tularemia protein fragments: Tfp, pilA, pilC, pilT, and pilQ attached to the aminoterminal end of the ecdCD40L to induce levels of anti-Tularemia antibodies that are completely protective of vaccinated individuals against dermal, oral or pulmonary acquisition of FT during the first year after the vaccination.
4. The administration of expression vectors (viral or DNA plasmid) vaccines which encode the following Tularemia protein fragments: Tfp, pilA, pilC, pilT, and pilQ attached to the aminoterminal end of the ecdCD40L to inactivate the function of the fimbriae or the pilus protein complex of FT and thereby prevent its infection of human cells and its subsequent replication within these cells.
5. The administration of expression vectors (viral or DNA plasmid) vaccines which encode the following Tularemia protein fragments: Tfp, pilA, pilC, pilT, and pilQ attached to the aminoterminal end of the ecdCD40L, which in contrast to the attenuated live Tularemia vaccine, have a much lower probability to lead to significant side effects in the vaccinated individuals.
6. The administration of expression vectors (viral or DNA plasmid) vaccines which encode the following Tularemia protein fragments: Tfp, pilA, pilC, pilT, and pilQ attached to the aminoterminal end of the ecdCD40L to induce memory cells such that the protection against FT will last greater than one year.
7. The administration of expression vectors (viral or DNA plasmid) vaccines which encode the following Tularemia protein fragments: Tfp, pilA, pilC, pilT, and pilQ attached to the aminoterminal end of the ecdCD40L, which is contrast to the live attenuated FT vaccines, does not carry the risk of revision to a more virulent form of FT which could produce serious organ toxicity or death of the vaccinated individuals.

### Final Formulation of a Preferred Embodiment Vaccine

The vaccine, in a preferred embodiment, will be administered IM weekly for three weeks as a mixture of expression vectors (viral or plasmid) which encode the following fusion proteins:
1. The Tfp/ecdCD40L Vaccine. This vaccine consists of an expression vector (viral or plasmid cDNA) which encodes a fusion protein comprised of (from the aminoterminal end to the carboxyterminal end): a consensus sequence from the very amino terminal tip of the fimbriae or pilus that is formed by the Tfp proteins linked to the ecd of the CD40L. This vaccine will be designated as tfp/ecdCD40L.
2. The pilA/ecdCD40L Vaccine. This vaccine consists of an expression vector (viral or plasmid cDNA) which encodes a fusion protein comprised of (from the aminoterminal end to the carboxyterminal end): a fragment of the pilA protein linked to the ecd of the CD40L. This vaccine will be designated as pilA/ecdCD4L.
3. The pilC/ecdCD40L Vaccine. The pilC protein is important in the secretion of the Tfp complex. This vaccine consists of an expression vector (viral or plasmid cDNA) which encodes a fusion protein comprised of (from the aminoterminal end to the carboxyterminal end): a fragment of the pilC protein linked to the ecd of the CD40L. This vaccine will be designated as pilC/ecdCD40L.
4. The pilT/ecdCD40L Vaccine. The pilT protein is important for the virulence of FT strains. It is known to be an ATPase (13) but it is not clear that its virulence in FT involves its ATPase function. This vaccine consists of an expression vector (viral or plasmid cDNA) which encodes a fusion protein comprised of (from the aminoterminal end to the carboxyterminal end): a fragment of the pilT protein linked to the ecd of the CD40L. This vaccine will be designated as pilT/ecdCD40L.
5. The pilQ/ecdCD40L Vaccine. The pilQ protein is important in the secretion of the Tfp complex. This vaccine consists of an expression vector (viral or plasmid cDNA) which encodes a fusion protein comprised of (from the aminoterminal end to the carboxyterminal end): a fragment of the pilQ protein linked to the ecdCD40L. This vaccine will be designated as pilQ/ecdCD40L.

### REFERENCES

1. Loegering D J, Drake J R, Banas J A, McNealy T L, McArthur D G, Webster L M, and Lennartz M R. Francisella tularensis LVS grown in macrophages has reduced ability to stimulate the secretion of inflammatory cytokines by macrophages in vitro. Microb Pathog 41: 218-225, (2006).
2. Carlson Jr, P E, Carroll J A, O'Dee D M, and Nau G J. Modulation of virulence factors in Francisella tularensis determines human macrophage responses. Microb Pathog 42: 204-214, (2007).
3. Telepnev M, Golovilov I, Grundstrom T, Tarnvik A, and Sjostedt A. Francisella tularensis inhibits Toll-like receptor-mediated activation of intracellular signaling and secretion of TNF-alpha and IL-1 from murine macrophages. Cellular Microbiology 5: 41-51, (2003).
4. Bosio C M and Dow S W. Francisella tularensis induces aberrant activation of pulmonary dendritic cells. Journal of Immunology 175: 6792-6801, (2005).
5. McCrumb Jr F R. Aerosol infection of man with Pasteurella tularensis. Bacteriol Rev 25: 262-267, (1961).
6. Kavanaugh C N. Tularemia: a consideration of 123 cases with observations at autopsy. AMA Arch Internal Med 55: 61-85, (1935).
7. Homick R B and Eigelbach H T. Aerogenic immunization of man with live tularemia vaccine. Bacteriological Reviews 30: 532-538, (1966).
8. Conlan J W. Tularemia vaccines: recent developments and remaining hurdles. Review Future Microbiology 6: 391-405, (2011).
9. Clemens D L, Lee B Y, and Horwitz M A. Francisella tularensis enters macrophages via a novel process involving pseudopod loops. Infection and Immunity 73: 5892-5902, (2005).
10. Chong A, Wehrly T D, Nair V, Fischer E R, Barker J R, Klose K E, and Celli J. The early phagosomal stage of Francisella tularensis determines optimal phagosomal escape and Francisella pathogeniscity island protein expression. Infection and Immunity 76: 5488-5499, (2009).
11. Meibom K L, and Charbit A. The unraveling panoply of Francisella tularensis virulence attributes. Current Opinion in Microbiology 13: 11-17, (2010).
12. Chakraborty S, Monfett M, Maier T M, Benach J L, Frank D W, and Thanassi DG. Type IV Pili in Francisella tularensis: roles of pilF and pilT in fiber assembly, host cell adherence and virulence. Infection and Immunity 76: 2852-2861, (2008).
13. Forslund A L, Salomonsson E N, Golvliov I, Kjoppa K, Michell S, Titball R, Oyston P, Noppa L, Sjostedt A, and Forsberg A. The type IV pilin, pilA, is required for full virulence of Francisella tularensis subspecies tularensis. BMC Microbiology 10: 227-234, (2010).
14. Zhang, L, Tang, Y, Akbulut H, Zelterman D, Linton P-J, and Deisseroth, A. An adenoviral vector cancer vaccine that delivers a tumor-associated antigen/CD40-ligand fusion protein to dendritic cells. PNAS, 100: 15101-15106, (2003).
15. Akbulut, H, Tang, Y, Maynard J, Zhang L, Pizzorno G, and Deisseroth, A. Vector targeting makes 5-fluorouracil chemotherapy less toxic and more effective in animal models of epithelial neoplasms. Clin Cancer Res 10: 7738-7746, (2004).
16. Tang, Y, Zhang, L, Yuan, J, Akbulut H, Maynard J, Linton P-J, and Deisseroth, A. Multistep process through which adenoviral vector vaccine overcomes anergy to tumor-associated antigens. Blood, 104: 2704-2713, (2004).
17. Akbulut H, Tang Y C, Akbulut K G, Maynard J, Zhang L, Deisseroth A. Antitumor immune response induced by i.t. injection of vector activated dendritic cells and chemotherapy suppresses metastatic breast cancer. Mol Cancer Ther 5:1975-1985, (2006).
18. Tang Y C, Maynard J, Akbulut H, Fang X M, Zhang W W, Xia X Q, Koziol J, Linton P-J, and Deisseroth A. Vaccine which overcomes defects acquired during aging and cancer. Journal of Immunology 177:5697-5707, (2006).
19. Tang Y, Akbulut H, Maynard J, Zhang L, Petersen L, and Deisseroth A. Vaccine strategies for cancer and infectious diseases in the elderly. Gene Therapy, Eds. Takenori Ochiai, Hideaki Shimada, and Masatoshi Tagawa, Published by Japanese Ministry of Education and Science, pp. 78-85, (2007).
20. Akbulut H, Akbulut K G, Tang Y C, Maynard J and Deisseroth A. Chemotherapy targeted to cancer tissue potentiates antigen specific immune response induced by vaccine for In vivo antigen loading and activation of dendritic cells. Molecular Therapy, 10:1753-1760, (2008).
21. Tang, Y C, Linton, P J, Thoman M, and Deisseroth A. Symposium in Writing: Vaccine for infections and cancer. Cancer Immunology and Immunotherapy, 58: 1949-1957, (2009).
22. Han T H, Tang, Y C, Park Y H, Petersen L, Maynard J, Li P C, and Deisseroth A. Ad-sig-BcrAbl/ecdCD40L vector prime-BcrAbl/ecdCD40L protein boost vaccine for P210Bcr-Abl protein. Bone Marrow Transplantation, (2009).
23. Akbulut H, Tang Y, Akbulut K G, Maynard J, and Deisseroth A. Addition of adenoviral vector targeting of chemotherapy to the MUC-1/ecdCD40L VPPP vector prime protein boost vaccine prolongs survival of mice carrying growing subcutaneous deposits of Lewis lung cancer cells. Gene Therapy, 17: 1333-1340, (2010).
24. Deisseroth A, Tang Y, Zhang L, Akbulut H, and Habib N. TAA/ecdCD40L adenoviral prime-protein boost vaccine for cancer and infectious diseases. Cancer Gene Therapy 20: 65-69, (2013).
25. Gil H, Benach J L, and Thanassi D G. Presence of pili on the surface of Francisella tularensis. Infection and Immunity 72: 3042-3047, (2004).
26. Forslund A, Kuoppa K, Svensson K, Salomonsson E, Johansson A, et al. Direct repeat-nediated deletion of a type IV pilin gene results in major virulence attenuation of Francisella tularensis. Mol Microbiol 59: 1818-1830, (2006).
27. Salomonsson E, Kuoppa K, Forslund A, Zingmark C, Golovliov I et al. Reintroduction of two deleted virulence loci restores full virulence to the live vaccine strain of Francisella tularensis. Infect Immun 77: 3424-3431, (2009).
28. Strom M, and Lory S. Structure-function and biogenesis of the type IV pili. Annual Review Microbiol 47: 565-596, (1993).
29. Hager A J, Bolton D L, Pelletier M R, Brittnacher M J, Gallagher L A, Kal R, Skerrett S J, Miller S I, and Guina T. Tupe IV pili-mediated secretion modulates Francisella virulence. Mol Microbiol 62: 227-237, (2006).
30. Mattick J. Type IV pili and twitching motility. Annual Rev Microbiol 56: 289-314, (2002).
31. Jefferson T et al. Efficacy and effect of influenza vaccines in the elderly. Lancet 264: 1165-1174, (2005).
32. Goodwin K, Vibou C, Simonsen L. Antibody response to influenza vaccination in the elderly: a quantitative review. Vaccine 24: 1159-1169, (2006).
33. Jackson M L, Nelson J C, Weiss N S et al. Influenza vaccination and risk of community acquired pneumonia in immunocompetent elderly people: a population based nested case control study. The Lancet 372: 398-405, (2008).
34. Simonsen L, Taylor R J. Mortality benefits of influenza vaccination in elderly people: an ongoing controversy Lancet Infect Dis 7: 658-666, 2007., Kraus A A, Haymore L B et al. Dengue virus neutralization by human immune sera: role of envelope protein domain III-reactive antibody. Virology 392: 103-113, (2009).

1. A method of generating an immune response in an individual against a Tularemia bacterial cell infection, by administering to the individual an effective amount of a multi-fragment vaccine combination each vaccine comprising an expression vector comprising a transcription unit encoding a secretable fusion protein comprising at least one fragment from each of five Tularemia proteins comprising Tfp, pilA, pilC, pilT, and pilQ, wherein each of said fragments is separately linked to an aminoterminal end of an ecd CD40 ligand to define said multi-fragment vaccine combination,
wherein each of said fragments (i) contains aminoacid domains which bind to and are recognized by Class I MHC, (ii) contains aminoacid domains which bind to and are recognized by Class II MHC, (iii) is small enough so as not to disrupt the homotrimeric structure of the ecdCD40L, (iv) contains a biological functional capability to block in each protein a functional capability of the Tularemia necessary for infection, and
whereby said five fragments each forming a part of one of five distinct vaccines are simultaneously administered to define a single vaccination combination adapted to decrease the probability of immunological escape.

2. A method according to claim 1, wherein each of said fragments is adapted to generate antibodies and CD8 effector T cells against the Tularemia infection.

3. A method according to claim 2, wherein each of said five fragments is selected to contain a virulence characteristic such that if virulence is a property of the bacterial cell, the severity of an infection would be reduced.

4. A method according to claim 1, wherein, a plasmid is employed instead of said expression vector for administering the multi-fragment vaccine combination subcutaneously or subdermally to the individual.

5. A pharmaceutical composition for generating an immune response in an individual against a Tularemia bacterial cell infection, by administering to the individual an effective amount of a multi-fragment vaccine combination each vaccine comprising an expression vector comprising a transcription unit encoding a secretable fusion protein comprising at least one fragment from each of five proteins comprising Tfp, pilA, pilC, pilT, and pilQ, wherein each of said fragments is linked to an aminoterminal end of an ecd CD40 ligand to define said multi-fragment vaccine combination,
wherein each of said fragments (i) contains aminoacid domains which bind to and are recognized by Class I MHC, (ii) contains aminoacid domains which bind to and are recognized by Class II MHC, (iii) is small enough so as not to disrupt the homotrimeric structure of the ecdCD40L, (iv) contains a biological functional capability capable to block in each protein a functional capability of the Tularemia necessary for infection, and
wherein said five fragments each forming a part of one of five distinct vaccines, are adapted when simultaneously administered to define a single vaccination combination to decrease the probability of immunological escape.

6. The composition of claim 5, wherein each of said five fragments is selected to contain a virulence characteristic such that if virulence is a property of the bacterial cell the severity of a Tularemia infection would be reduced.

7. The composition of claim 5, wherein each of said fragments is adapted to generate antibodies and CD8 effector T cells against the Tularemia infection.

8. The composition of claim 5, wherein a plasmid is employed instead of said expression vector for administering the multi-fragment vaccine subcutaneously or subdermally to the individual.

9. The composition of claim 5, wherein said five vaccines are mixed together to form a single vaccine.
